(19) United States
(12) Patent Application Publication
(10) Pub. No.: US 2004/0133993 A1
(43) Pub. Date: Jul. 15, 2004

(54) Oxidation dyeing composition for keratin fibers comprising a cationic poly(vinyllactam) and at least one oxidation dye in the form of a sulphate ion

(76) Inventors: Francois Cottard, Courbevoie (FR); Christine Rondeau, Sartrouville (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/688,958

(22) Filed: Oct. 21, 2003

Related U.S. Application Data
(60) Provisional application No. 60/475,490, filed on Jun. 4, 2003.

(30) Foreign Application Priority Data
Oct. 21, 2002 (FR) 02 13102

(51) Int. Cl. A61K 7/13

(52) U.S. Cl. 8/405

(57) ABSTRACT

The invention relates to a composition for the oxidation dyeing of keratin fibers, such as human keratin fibers, comprising, in a medium suitable for dyeing, at least one oxidation dye in the form of a sulphate salt and at least one cationic poly(vinyllactam).

The invention also relates to the dyeing methods and devices using the composition.

This application claims benefit of U.S. Provisional Application No. 60/475,490, filed Jun. 4, 2003.

The present invention relates to a composition for the oxidation dyeing of keratin fibers, in particular human keratin fibers such as hair, comprising at least one oxidation dye in the form of a sulphate salt and at least one cationic poly(vinyllactam).

It is known to dye keratin fibers, and in particular human hair, with dyeing compositions comprising oxidation dye precursors, generally called “oxidation bases”, such as, for example, ortho- or para-phenylenediamines, ortho- or para-aminophenols, and heterocyclic bases.

Oxidation dye precursors may be compounds initially only slightly colored or not colored and which develop dyeing power in the presence of oxidizing agents, leading to the formation of colored compounds. The formation of these colored compounds may result either from oxidative condensation of the “oxidation bases” with themselves, or oxidative condensation of the “oxidation bases” with color modifying compounds, or “couplers”, which are generally present in the dyeing compositions used in oxidation dyeing and can be, for example, meta-phenylenediamines, meta-aminophenols, meta-diphenols, and certain heterocyclic compounds.

The variety of molecules used, which comprise, on the one hand, “oxidation bases” and on the other hand, “couplers”, may allow a very rich palette of colors to be obtained.

To confine the oxidation dyeing product upon application to the hair so that it does not run over the face or outside the areas which it is desired to dye, use has up until now been made of traditional thickeners such as crosslinked polyacrylic acid, hydroxyethyl-celluloses, certain polyurethanes, waxes or mixtures of nonionic surfactants having an HLB (Hydrophilic Lipophilic Balance), suitably chosen, which produce a gelling effect when they are diluted with water and/or surfactants.

Many of the thickening systems of the prior art do not make it possible to obtain intense and chromatic shades of low selectivity and good fastness, nor do they offer a good cosmetic condition to the treated hair. Moreover, it has been observed that many of the ready-to-use dyeing compositions of the prior art containing at least one oxidation dye and a thickening system do not allow a sufficiently precise application without running or a decrease in viscosity over time.

French Patent Application 2 820 032 describes ready-to-use oxidation dyeing compositions which do not run and therefore remain well confined to the site of application. These compositions of French Patent Application 2 820 032 comprise, in a medium suitable for dyeing, at least one oxidation dye and at least one cationic poly(vinyllactam). The compositions also make it possible to obtain intense and chromatic, i.e., radiant, shades with low selectivities and good fastness towards chemical agents, such as shampoos, permanent waving chemicals, and the like, or natural agents, such as light, perspiration, and the like, while offering the hair good cosmetic properties.

The compositions comprising at least one oxidation dye and a thickening system may be provided in the form of creams. The current technology in the oxidation dye field then requires that these compositions, in order to acquire a cream appearance, comprise high contents of fatty active agents, such as alcohols, amides, and acids.

However, the present inventors have observed that as the viscosity of these creams changes during storage, it is difficult to obtain a homogeneous mixture when these compositions are mixed in the form of a cream with an oxidizing agent. In addition, the consistency of these creams may make them difficult to use.

Furthermore, it has been discovered that the dyeing compositions in the form of creams containing high concentrations of oxidation dyes in the form of salts, such as sulphate salts, are generally not stable.

The present inventors have discovered, surprisingly, that it is possible to obtain ready-to-use oxidation dyeing compositions, rich in dye in the form of sulphate salts, which may exhibit an increased ease of mixing with at least one oxidizing agent and other optional components, an improvement in the foaming properties, and an increased ease of elimination, such as during rinsing.

In addition, the compositions disclosed herein may not run and therefore may remain well confined to the site of application. The compositions disclosed herein also may make it possible to obtain intense and chromatic, i.e., radiant, shades with low selectivities and good fastness towards chemical agents, such as shampoos, permanent waving chemicals, and the like, or natural agents, such as light, perspiration, and the like, while offering the hair good cosmetic properties.

It has also been observed that the compositions disclosed herein can have reduced contents of fatty active agents compared with the contents of prior art compositions without the consistency of the composition (cream) being affected.

The present inventors have also shown that it is possible to obtain stable compositions in the form of creams, for example comprising at least 2% by weight relative to the total weight of the composition of at least one oxidation dye in the form of a sulphate salt.

One embodiment disclosed herein is thus a composition for the oxidation dyeing of keratin fibers, in particular human keratin fibers, and in particular hair, comprising, in a medium suitable for dyeing, at least one oxidation dye in the form of a sulphate salt, for example in a concentration of at least 2% by weight relative to the total weight of the composition, and at least one cationic poly(vinyllactam).

Another embodiment disclosed herein is a ready-to-use composition for dyeing keratin fibers which comprises at least one oxidation dye in the form of a sulphate salt, for example in a concentration of at least 2% by weight relative to the total weight of the composition, at least one cationic poly(vinyllactam) as defined below, and at least one oxidizing agent.

The expression “ready-to-use composition” is understood to mean, for the purposes of the present invention, the composition intended to be applied as it is to keratin fibers, i.e., it can be stored as it is before use or be obtained from the mixing of two or more compositions.

An effective quantity of cationic poly(vinyllactam) is thus introduced:

(A) into at least one composition A comprising at least one oxidation dye and optionally comprising at least one coupler; or

(B) into at least one oxidizing composition B; or

(C) into both compositions A and B at the same time.

Disclosed herein is also a method for dyeing keratin fibers, such as human keratin fibers, such as hair, comprising applying to the fibers at least one composition A comprising, in a medium suitable for dyeing, at least one oxidation dye in the form of a sulphate salt, for example in a concentration of at least 2% by weight relative to the total weight of the composition, wherein the color is developed at alkaline, neutral or acidic pH with the aid of at least one oxidizing composition (composition B)comprising at least one oxidizing agent which is mixed at the time of use with the at least one composition A or which is applied sequentially with the at least one composition A without intermediate rinsing, and wherein at least one cationic poly(vinyllactam) as defined below is present in the at least one composition A and/or in the at least one composition B.

Another embodiment disclosed herein is a multicompartment dyeing device or “kit”.

For example, a 2-compartment device as disclosed herein may comprise a first compartment containing at least one composition Al comprising, in a medium suitable for dyeing, at least one oxidation dye in the form of a sulphate salt, for example in a concentration of at least 2% by weight relative to the total weight of the composition, and a second compartment containing at least one composition B1 comprising, in a medium suitable for dyeing, at least one oxidizing agent, wherein at least one cationic poly(vinyllactam) polymer as defined below is present in the at least one composition A1 and/or the at least one composition B1.

As a further example, a 3-compartment device as disclosed herein may comprise a first compartment containing at least one composition A2 comprising, in a medium suitable for dyeing, at least one oxidation dye in the form of a sulphate salt, for example in a concentration of at least 2% by weight relative to the total weight of the composition, a second compartment containing at least one composition B2 comprising, in a medium suitable for dyeing, at least one oxidizing agent, and a third compartment containing at least one composition C comprising, in a medium suitable for dyeing, at least one cationic poly(vinyllactam) polymer, it also being possible for the at least one composition A2 and/or the at least one composition B2 to comprise at least one cationic poly(vinyllactam) polymer as defined below.

Other characteristics, aspects, subjects, and advantages of the invention will appear more clearly on reading the description and the examples which follow.

Associative polymers are polymers whose molecules are capable, in the formulation medium, of combining with each other or with molecules of other compounds.

The chemical structure of associate polymers generally comprises at least one hydrophilic region and at least one hydrophobic region, the at least one hydrophobic region comprising at least one fatty chain.

Cationic Poly(Vinyllactam) Polymers According to the Invention

The cationic poly(vinyllactam) polymers disclosed herein comprise:

a) at least one monomer chosen from vinyllactams and alkylvinyllactams; and

b) at least one monomer chosen from the following structures (Ia) and (Ib):

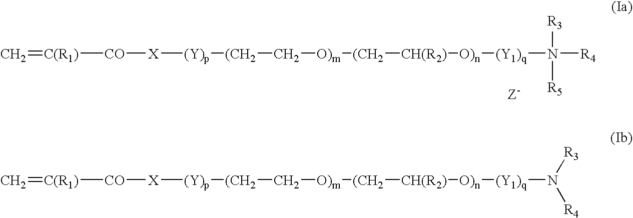

in which:

X is chosen from oxygen atoms and NR₆ radicals;

R₁ and R₆, which may be identical or different, are chosen from hydrogen atoms, linear C₁-C₅ alkyl radicals, and branched C₁-C₅ alkyl radicals,

R₂ is chosen from linear C₁-C₄ alkyl radicals and branched C₁-C₄ alkyl radicals,

R₃, R₄ and R₅, which may be identical or different, are chosen from hydrogen atoms, linear C₁-C₃₀ alkyl radicals, branched C₁-C₃₀ alkyl radicals, and radicals of formula (II):

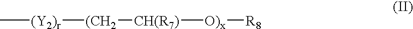

wherein

—Y, Y₁ and Y₂, which may be identical or different, are chosen from linear C₂-C₁₆ alkylene radicals and branched C₂-C₁₆ alkylene radicals,

R₇ is chosen from hydrogen atoms, linear C₁-C₄ alkyl radicals, branched C₁-C₄ alkyl radicals, linear C₁-C₄ hydroxyalkyl radicals, and branched C₁-C₄ hydroxyalkyl radicals,

R₈ is chosen from hydrogen atoms, linear C₁-C₃₀ alkyl radicals, and branched C₁-C₃₀ alkyl radicals,

p, q and r, which may be identical or different, are chosen from 0 or 1,

m and n, which may be identical or different, are chosen from integers ranging from 0 to 100,

x is an integer ranging from 1 to 100,

Z⁻ is chosen from organic acid anions and inorganic acid anions,

with the provisos that:

at least one of R₃, R₄, R₅ or R₈ is chosen from linear C₉-C₃₀ alkyl radicals and branched C₉-C₃₀ alkyl radicals,

if either m or n is different from 0, then q is 1,

if either m or n is 0, then either p or q is 0.

The at least one cationic poly(vinyllactam) polymer disclosed herein may be crosslinked or noncrosslinked and may also be block polymers.

In certain embodiments, the counterion Z⁻ of the monomers of formula (Ia) is chosen from halide ions, phosphate ions, methosulphate ions, and tosylate ions.

In some embodiments, R₃, R₄ and R₅, which may be identical or different, are chosen from hydrogen atoms, linear C₁-C₃₀ alkyl radicals, and branched C₁-C₃₀ alkyl radicals.

In certain embodiments, the monomer (b) is at least one monomer of formula (Ia) for which, for example, m and n are 0.

The at least one monomer chosen from vinyllactams and alkylvinyllactams is, for example, a compound having the structure (III):

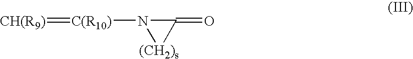

in which:

s is an integer ranging from 3 to 6,

R₉ is chosen from hydrogen atoms and C₁-C₅ alkyl radicals,

R₁₀ is chosen from hydrogen atoms and C₁-C₅ alkyl radicals,

provided that at least one of R₉ and R₁₀ is a hydrogen atom.

In at least one embodiment, the monomer (III) is vinylpyrrolidone.

The cationic poly(vinyllactam) polymers disclosed herein may further comprise at least one additional monomer, such as cationic monomers or nonionic monomers.

As compounds which may be useful herein, mention may be made of the following terpolymers comprising:

a) at least one monomer (a) of formula (III);

b) at least one monomer (b) of formula (Ia) in which p is 1, q is 0, R₃ and R₄, which may be identical or different, are chosen from hydrogen atoms and C₁-C₅ alkyl radicals, and R₅ is a C₉-C₂₄ alkyl radical; and

c) at least one monomer (c) of formula (Ib) in which R₃ and R₄, which may be identical or different, are chosen from hydrogen atoms and C₁-C₅ alkyl radicals.

In yet another embodiment, as useful compounds may be mentioned terpolymers comprising, by weight relative to the total weight of the terpolymer, 40% to 95% of the at least one monomer (a), 0.1% to 55% of the at least one monomer (c), and 0.25% to 50% of the at least one monomer (b) . Such polymers are described, for example, in patent application WO-00/68282, the disclosure of which is incorporated by reference herein.

As cationic poly(vinyllactam) polymers disclosed herein, mention may be made, for example, of the following terpolymers:

vinylpyrrolidone/dimethylaminopropylmethacrylamide/dodecyldimethylmethacrylamidopropylammonium tosylate,

vinylpyrrolidone/dimethylaminopropylmethacrylamide/cocoyidimethylmethacrylamidopropylammonium tosylate, and

vinylpyrrolidone/dimethylaminopropylmethacrylamide/lauryldimethylmeth-acrylamidopropylammonium tosylate or chloride.

The weight-average molecular mass of the cationic poly(vinyllactam) polymers may range, for example, from 500 to 20,000,000. For example, it may range from 200,000 to 2,000,000 , such as from 400 000 to 800 000.

In the dyeing compositions disclosed herein, the at least one cationic poly(vinyllactam) described above may be used in an amount which may range from 0.01% to 10% by weight relative to the total weight of the composition. For example, this quantity may range from 0.1% to 5% by weight relative to the total weight of the composition.

In certain embodiments, the viscosity of these compositions may be at least 1 000 cp, measured at 25° C. using a RHEOMAT RM 180 rheometer at the shear rate of 200 s⁻¹.

Oxidation Dyes in the Form of Sulphate Salts

The compositions disclosed herein comprise at least one oxidation dye in the form of a sulphate salt, for example in a concentration of at least 2% by weight relative to the total weight of the composition. This oxidation dye is chosen from bases and couplers in the form of conventionally used sulphate salts.

These sulphate salts include, for example, sulphate salts of oxidation bases such as para-phenylenediamines, double bases, ortho- and para-aminophenols, and heterocyclic bases, as well as sulphate salts of couplers such as meta-aminophenols, meta-phenylenediamines, meta-diphenols, naphthols, and heterocylic couplers.

As a further example, the sulphate salts may be chosen from para-toluenediamine sulphate, N,N-bis(β-hydroxyethyl)-para-phenylenediamine sulphate, 2-(β-hydroxyethyl)-para-phenylenediamine sulphate and N-methyl-para-aminophenol hemisulphate, 1-(β-hydroxyethyl)-4,5-diaminopyrazole sulphate, and 4-(β-hydroxyethyl)amino-2-aminoanisole sulphate.

In the dyeing compositions disclosed herein, the at least one oxidation dye in the form of a sulphate salt may be, for example, used in an amount ranging from 2% to 25% by weight relative to the total weight of the composition. In another embodiment, the amount ranges from 2.25% to 15% by weight relative to the total weight of the composition, and in still another embodiment, the amount ranges from 2.5% to 10% by weight relative to the total weight of the composition.

Oxidation Dyes

The compositions disclosed herein may also comprise at least one additional oxidation dye other than the at least one oxidation dye in the form of a sulphate salt. The at least one additional oxidation dye may be chosen from additional oxidation bases and additional couplers.

The additional oxidation bases which can be used herein may be chosen from those conventionally known in oxidation dyeing, and among which mention may be made, for example, of para-phenylenediamines, double bases, ortho- and para-aminophenols, and the following heterocyclic bases, as well as their acid addition salts other than sulphates.

Mention may be made, for example, of:

(A) the para-phenylenediamines of the following formula (IV) and their acid addition salts other than sulphates:

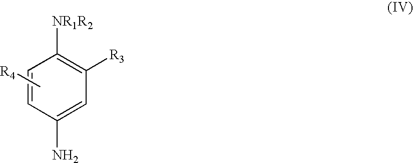

in which:

R₁ is chosen from hydrogen atoms, C₁-C₄ alkyl radicals, monohydroxy(C₁-C₄ alkyl) radicals, polyhydroxy(C₂-C₄ alkyl) radicals, (Cl-C₄)alkoxy(C₁-C₄)alkyl radicals, C₁-C₄ alkyl radicals optionally substituted with at least one nitrogen-containing group, phenyl radicals, and 4′-aminophenyl radicals;

R₂ is chosen from hydrogen atoms, C₁-C₄ alkyl radicals, monohydroxy(C₁-C₄ alkyl) radicals, polyhydroxy(C₂-C₄ alkyl) radicals, (C₁-C₄)alkoxy(C₁-C₄)alkyl radicals, and C₁-C₄ alkyl radicals optionally substituted with at least one nitrogen-containing group;

R₁ and R₂ may also form, with the nitrogen atom carrying them, a 5- or 6-membered nitrogen-containing heterocycle optionally substituted with at least one group chosen from alkyl, hydroxyl, and ureido groups;

R₃ is chosen from hydrogen atoms, halogen atoms such as chlorine atoms, C₁-C₄ alkyl radicals, sulpho radicals, carboxyl radicals, monohydroxy(C₁-C₄ alkyl) radicals, hydroxy(C₁-C₄ alkoxy) radicals, acetylamino(C₁-C₄ alkoxy) radicals, mesylamino(C₁-C₄ alkoxy) radicals, and carbamoylamino(C₁-C₄ alkoxy) radicals; and

R₄ is chosen from hydrogen atoms, halogen atoms, and C₁-C₄ alkyl radicals.

Among the nitrogen-containing groups of formula (IV) above, mention may be made of amino, mono(C₁-C₄)alkylamino, (C₁-C₄)dialkylamino, (C₁-C₄)trialkylamino, monohydroxy(C₁-C₄)alkylamino, imidazolinium and ammonium radicals.

Among the para-phenylenediamines of formula (IV) above, mention may be made of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-methyl-1-N-β-hydroxyethyl-para-phenylenediamine and their acid addition salts other than sulphates.

For example, among the para-phenylenediamines of formula (IV) listed above, mention may be made of para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and their acid addition salts other than sulphates.

(B) According to the invention, “double bases” is understood to mean the compounds containing at least two aromatic rings on each of which at least one amino and/or at least one hydroxyl group is carried.

Among the double bases which can be used as additional oxidation bases in the dyeing compositions disclosed herein, mention may be made of the compounds corresponding to the following formula (V), and their acid addition salts other than sulphates:

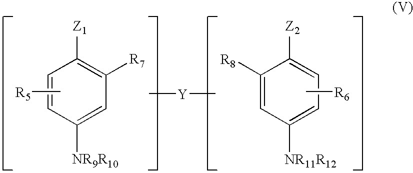

in which:

Z₁ and Z₂, which may be identical or different, are chosen from hydroxyl radicals and

NH₂ radicals, optionally substituted with at least one C₁-C₄ alkyl radical or with a linking arm Y;

the linking arm Y is chosen from linear alkylene chains or branched alkylene chains comprising 1 to 14 carbon atoms, optionally interrupted by or optionally ending with at least one nitrogen-containing group and/or at least one heteroatom such as oxygen, sulphur or nitrogen atoms, and optionally substituted with at least one radical chosen from hydroxyl radicals and C₁-C₆ alkoxy radicals;

R₅ and R₆ are chosen from hydrogen atoms, halogen atoms, C₁-C₄ alkyl radicals, monohydroxy(C₁-C₄ alkyl) radicals, polyhydroxy(C₂-C₄ alkyl) radicals, amino(C₁-C₄ alkyl) radicals, and linking arms Y;

R₇, R₈, R₉, R₁₀, R₁₁ and R₁₂, which may be identical or different, are chosen from hydrogen atoms, a linking arm Y, and C₁-C₄ alkyl radicals;

with the proviso that the compounds of formula (V) comprise only one linking arm Y per molecule.

Among the nitrogen-containing groups of formula (V) above, mention may be made of the amino, mono(C₁-C₄)alkylamino, (C₁-C₄)dialkylamino, (C₁-C₄)trialkylamino, monohydroxy(C₁-C₄)alkylamino, imidazolinium and ammonium radicals.

Among the double bases of formula (V) above, mention may be made of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetra methylenediamine, N,N′-bis(β-hydroxyethyl)-N,N′-bis(4-aminophenyl)tetra methylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their acid addition salts other than sulphates.

Among these double bases of formula (V), mention may be made of, for example, N,N′-bis(β-hydroxyethyl)-N,N′-bis(4′-aminophenyl)-1,3-diaminopropanol, 1,8-bis(2,5-diaminophenoxy)-3,6-dioxaoctane or one of their acid addition salts other than sulphates.

(C) the para-aminophenols corresponding to the following formula (VI), and their acid addition salts other than sulphates:

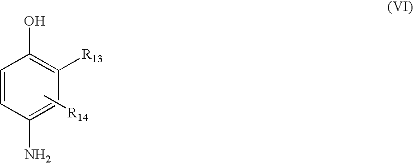

in which:

R₁₃ is chosen from hydrogen atoms, halogen atoms such as fluorine, C₁-C₄ alkyl radicals, monohydroxy(C₁-C₄ alkyl) radicals, (C₁-C₄)alkoxy(C₁-C₄)alkyl radicals, amino(C₁-C₄ alkyl) radicals, and hydroxy(C₁-C₄)alkylamino(C₁-C₄ alkyl) radicals,

R₁₄ is chosen from hydrogen atoms, halogen atoms such as fluorine, a C₁-C₄ alkyl radicals, monohydroxy(C₁-C₄ alkyl) radicals, polyhydroxy(C₂-C₄ alkyl) radicals, amino(C₁-C₄ alkyl) radicals, cyano(C₁-C₄ alkyl) radicals, and (C₁-C₄)alkoxy(C₁-C₄)alkyl radicals.

Among the para-aminophenols of formula (VI) above, mention may be made of, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, and their acid addition salts other than sulphates.

(D) the ortho-aminophenols which can be used as additional oxidation bases herein may be chosen from 2-aminophenol, 2-amino-1-hydroxy-5-methylbenzene, 2-amino-1-hydroxy-6-methylbenzene, 5-acetamido-2-aminophenol, and their acid addition salts other than sulphates.

(E) among the heterocyclic bases which can be used as additional oxidation bases in the dyeing compositions herein, mention may be made of pyridine derivatives, pyrimidine derivatives, pyrazole derivatives, and their acid addition salts other than sulphates.

Among the pyridine derivatives, mention may be made of the compounds described for example in Patents GB 1,026,978 and GB 1,153,196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diamino-pyridine, and their acid addition salts other than sulphates.

Among the pyrimidine derivatives, mention may be made of the compounds described for example in German Patent DE 2,359,399, Japanese Patents JP 88-169,571 and JP 91-10659, and Patent Applications WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the pyrazolopyrimidine derivatives such as those mentioned in Patent Application FR-A-2,750,048 and among which mention may be made of, for example, pyrazolo[1,5-a]pyrimidine-3,7-diamine, 2,5-dimethylpyrazolo[1,5-a]pyrimidine-3,7-diamine; pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-di-methylpyrazolo[1,5-a]pyrimidine-3,7-diamine, 2,6-dimethylpyrazolo[1,5-a]pyrimidine-3,7-diamine, 2,5,N7, N7-tetramethylpyrazolo[1,5-a]pyrimidine-3,7-diamine, 3-amino-5-methyl-7-imidazolylpropylaminopyrazolo[1,5-a]pyrimidine, and their acid addition salts other than sulphates and their tautomeric forms, when a tautomeric equilibrium exists.

Among the pyrazole derivatives, mention may be made of, for example, the compounds described in Patents DE 3,843,892, DE 4,133,957 and Patent Applications WO 94/08969, WO 94/08970, FR-A-2,733,749, and DE 195 43 988, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl )-3-methylpyrazole, 4,5-diamino-1-(β-hydroxyethyl)pyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their acid addition salts other than sulphates.

According to one embodiment disclosed herein, the additional oxidation bases may be present in an amount ranging from 0.0005% to 20% by weight relative to the total weight of the composition, for example, in an amount ranging from 0.005% to 8% by weight relative to the total weight of the composition.

The additional couplers which can be used in the dyeing method disclosed herein are those conventionally used in oxidation dyeing compositions, for example, meta-aminophenols, meta-phenylenediamines, meta-diphenols, naphthols and heterocyclic couplers such as, for example, indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles, quinolines and their acid addition salts other than sulphates.

These couplers may, for example, be chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole, and their acid addition salts other than sulphates.

When they are present, these additional couplers may be present in an amount ranging from 0.0001% to 20% by weight relative to the total weight of the composition, or, for example, from 0.005% to 5% by weight relative to the total weight of the composition.

In general, the acid addition salts of the additional oxidation bases and additional couplers may, for example, be chosen from hydrochlorides, hydrobromides, tartrates, lactates, and acetates.

The compositions disclosed herein may also comprise, in addition to the oxidation dyes defined above, direct dyes that may enrich the shades with glints. These direct dyes may, for example, be chosen from neutral, cationic or anionic nitro dyes, azo dyes, and anthraquinone dyes, in an amount ranging from 0.001% to 20% by weight relative to the total weight of the composition, for example, from 0.01% to 10% by weight relative to the total weight of the composition.

The composition A and/or the composition B and/or the composition C as disclosed above may additionally comprise at least one amphoteric polymer or at least one cationic polymer different from the cationic poly(vinyllactams) disclosed herein.

Cationic Polymers Different from the Cationic Poly(vinyllactams) Disclosed Herein

As used herein, the expression “cationic polymer” denotes any polymer comprising at least one cationic group and/or at least one group which can be ionized to at least one cationic group.

The cationic polymers which can be used herein may be chosen from all those already known per se to improve the cosmetic properties of hair, for example those described in Patent Application EP-A-337 354 and in French patents FR-2,270,846, 2,383,660, 2,598,611, 2,470,596 and 2,519,863.

Certain cationic polymers are chosen from those which comprise at least one unit comprising at least one amino group chosen from primary, secondary, tertiary and quaternary amino groups, which may either form part of the principal polymeric chain, or which may be carried by a side substituent linked directly thereto.

The cationic polymers used generally have a number-average molecular mass ranging from 500 to 5×10⁶, for example ranging from 10³ to 3.10⁶.

Among the cationic polymers, mention may be made of the polymers chosen from polyamines, polyamino amides, and quaternary polyammoniums.

These may be known products, for example those described in French patent Nos. 2,505,348 and 2,542,997. Among such cationic polymers, mention may be made of:

(1) the homopolymers or copolymers derived from the group chosen from acrylics, methacrylic esters, and amides, and comprising at least one of the units of the following formulae (VII), (VIII), (IX) or (X):

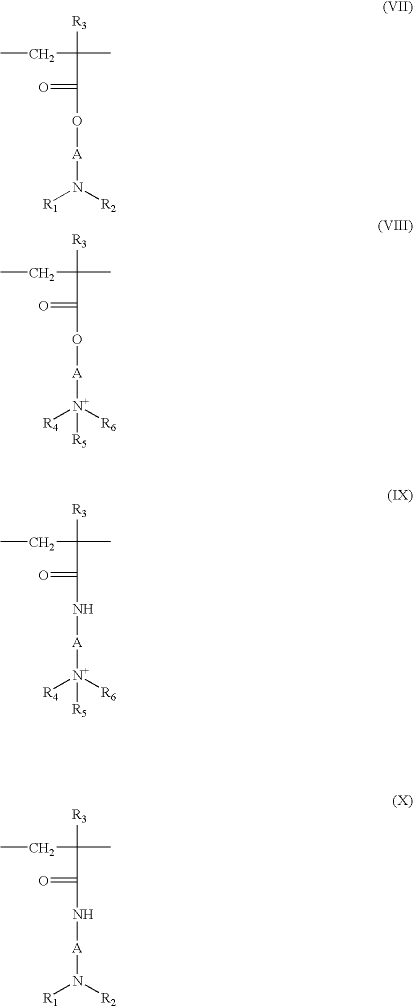

in which:

R₃, which may be identical or different, is chosen from hydrogen atoms and CH₃ radicals;

A, which may be identical or different, is chosen from linear or branched alkyl groups having 1 to 6 carbon atoms, for example 2 or 3 carbon atoms, and hydroxyalkyl groups having 1 to 4 carbon atoms;

R₄, R₅, and R₆, which may be identical or different, are chosen from alkyl groups having 1 to 18 carbon atoms, for example alkyl groups having 1 to 6 carbon atoms, and benzyl radicals;

R₁ and R₂, which may be identical or different, are chosen from hydrogen atoms and alkyl groups having from 1 to 6 carbon atoms, for example methyls and ethyls;

the charged species of formula (VIII) and (IX) are combined with a counterion X, wherein X is an anion derived from inorganic or organic acids such as methosulphate anions and halides such as chloride or bromide.

The polymers of the group (1) may comprise, in addition, at least one unit derived from comonomers which may be chosen from acrylamides, methacrylamides, and diacetone acrylamides, optionally substituted on the nitrogen with at least one group chosen from lower (C₁-C₄)alkyls, acrylic acids thereof, methacrylic acids thereof, acrylic esters thereof, methacrylic esters thereof, vinyllactams such as vinylpyrrolidone and vinylcaprolactam, and vinyl esters.

Thus, among these polymers of the group (1), mention may be made of:

the copolymers of acrylamide and dimethylamino-ethyl methacrylate quaternized with dimethyl sulphate or with a dimethyl halide such as that sold under the name HERCOFLOC® by the company HERCULES,

the copolymers of acrylamide and methacryloyloxy-ethyltrimethylammonium chloride described, for example, in Patent Application EP-A-080976 and sold under the name BINA QUAT P 100® by the company CIBA GEIGY,

the copolymers of acrylamide and methacryloyloxy-ethyltrimethylammonium methosulphate sold under the name RETEN® by the company HERCULES, the vinylpyrrolidone/dialkylaminoalkyl acrylate and methacrylate copolymers, optionally quaternized, such as the products sold under the name GAFQUAT® by the company ISP, such as for example GAFQUAT 734, GAFQUAT 755 or alternatively the products called COPOLYMER 845®, 958® and 937®. These polymers are described in detail in French Patents 2,077,143 and 2,393,573,

the dimethylaminoethyl methacrylate/vinylcapro-lactam/vinylpyrrolidone terpolymers such as the product sold under the name GAFFIX VC 713® by the company ISP,

the vinylpyrrolidone/methacrylamidopropyldimethyl-amine copolymers sold in particular under the name STYLEZE CC 10® by ISP,

and the quaternized vinylpyrrolidone/dimethyl-aminopropyl methacrylamide copolymers such as the product sold under the name GAFQUAT® HS 100 by the company ISP.

(2) The cellulose ether derivatives comprising quaternary ammonium groups, described in French Patent 1,492,597, and for example the polymers sold under the names JR®, such as JR 400, JR 125, and JR 30M, and LR®, such as LR 400 and LR 30M, by the Union Carbide Corporation. These polymers are also defined in the CTFA dictionary as hydroxyethyl cellulose quaternary ammoniums which have reacted with an epoxide substituted by a trimethylammonium group.

(3) Cationic cellulose derivatives such as cellulose copolymers and cellulose derivatives grafted with a quaternary ammonium water-soluble monomer, and described in U.S. Pat. No. 4,131,576, such as hydroxyalkyl celluloses such as hydroxymethyl, hydroxyethyl and hydroxypropyl celluloses grafted with at least one salt chosen from methacryloylethyltrimethylammonium, methacrylamidopropyltrimethylammonium and dimethyldiallylammonium salts.

The commercialized products corresponding to this definition are, for example, the products sold under the name Celquat® L 200 and Celquate H 100 by the company National Starch.

(4) The cationic polysaccharides described more particularly in U.S. Pat. Nos. 3,589,578 and 4,031,307 such as guar gums comprising cationic trialkylammonium groups. Guar gums modified with a 2,3-epoxypropyltrimethylammonium salt (e.g., chloride) are for example used.

Such products are sold, for example, under the trade names JAGUAR® C13 S, JAGUAR® C 15, JAGUAR® C 17 and JAGUAR® C162 by the company MEYHALL.

(5) Polymers comprising at least one of piperazinyl units, alkylene divalent radicals, and hydroxyalkylene divalent radicals, comprising straight or branched chains and optionally interrupted by at least one group chosen from oxygen atoms, sulphur atoms, nitrogen atoms, aromatic rings, heterocyclic rings, the oxidation products of these polymers, and the quaternization products of these polymers. Such polymers are described for example in French patents 2,162,025 and 2,280,361.

(6) Water-soluble polyaminoamides prepared for example by polycondensation of at least one acid compound with at least one polyamine, wherein these polyaminoamides may be crosslinked with at least one of epihalohydrins, diepoxides, dianhydrides, unsaturated dianhydrides, diunsaturated derivatives, bishalohydrins, bisazetidiniums, bishaloacyldiamines, and aalkylbishalides, or, for example, prepared by polycondensation of at least one acid compound with at least one oligomer resulting from the reaction of at least one difunctional compound which is reactive towards at least one of bishalohydrins, bisazetidiniums, bishaloacyldiamines, alkylbishalides, epihalohydrins, diepoxides and diunsaturated derivatives, wherein the the crosslinking agent may be employed in proportions ranging from 0.025 to 0.35 mol per amine group of the polyaminoamide. These polyaminoamides may be alkylated or, if they include at least one tertiary amine functional group, quaternized. Such polymers are described for example in French Patents 2,252,840 and 2,368,508.

(7) Polyaminoamide derivatives resulting, for example, from the condensation of polyalkylenepolyamines with polycarboxylic acids, followed by alkylation with difunctional agents. Mention may be made of, for example, adipic acid-dialkylaminohydroxyalkyl-dialkylenetriamine polymers in which the alkyl radical contains from 1 to 4 carbon atoms and, in some embodiments is chosen from methyls, ethyls and propyls. Such polymers are described for example in French Patent 1,583,363.

Among these derivatives mention may be made of adipic acid/dimethylaminohydroxypropyl/diethylenetriamine polymers sold under the name Cartaretine® F, F4 or F8 by the company Sandoz.

(8) Polymers obtained by reaction of at least one polyalkylenepolyamine comprising at least two primary amine groups and at least one secondary amine group with at least one dicarboxylic acid chosen from diglycolic acids and saturated aliphatic dicarboxylic acids having 3 to 8 carbon atoms. The molar ratio of the polyalkylenepolyamine to the dicarboxylic acid may, for example, range from 0.8:1 to 1.4:1, wherein the polyaminoamide resulting therefrom is made to react with epichlorohydrin in a molar ratio of epichlorohydrin relative to the secondary amine group of the polyaminoamide ranging from 0.5:1 to 1.8:1. Such polymers are described for example in U.S. Pat. Nos. 3,227,615 and 2,961,347.

Polymers of this type are sold, for example, under the name Hercosett® 57 by the company Hercules Inc. and under the name of PD 170® and Delsette 101® by the company Hercules in the case of copolymers of adipic acid/epoxypropyl/diethylene-triamine.

(9) Cyclopolymers of alkyldiallylamine or of dialkyldiallylammonium, such as the homopolymers and copolymers comprising, as main constituents of the chain, units corresponding to the formulae (XI) or (XII):

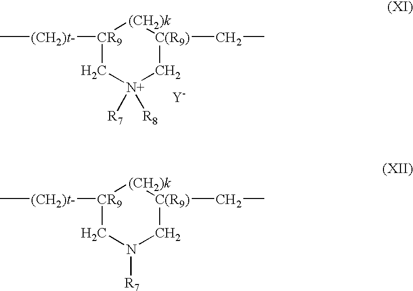

in which

k and t are chosen from 0 and 1, wherein the sum of k and t is 1;

R₉ is chosen from hydrogen atoms and methyl radicals;

R₇ and R8, which may be identical or different, are chosen from alkyl groups having 1 to 8 carbon atoms, hydroxyalkyl groups in which the alkyl groups, for example, have 1 to 5 carbon atoms, and lower (C₁-C₄)amidoalkyl groups; or

R₇ and R8 may form, together with the nitrogen atom to which they are attached, heterocyclic groups such as piperidinyl and morpholinyl; wherein

R₇ and R8, which may be identical or different, may for example be an alkyl group having 1 to 4 carbon atoms;

Y⁻ is an anion such as bromide, chloride, acetate, borate, citrate, tartrate, bisulphate, bisulphite, sulphate or phosphate.

These polymers are described for example in French Patent 2,080,759 and in its certificate of addition 2,190,406.

Among the polymers defined above mention may be made of dimethyldiallylammonium chloride homopolymer sold under the name Merquat® 100 by the company Calgon, as well as its homologues of low weight-average molecular masses, and copolymers of diallyl-dimethylammonium chloride and acrylamide sold under the name MERQUAT® 550.

(10) The quaternary diammonium polymer comprising repeating units corresponding to the formula (XIII):

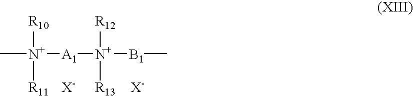

in which:

R₁₀, R₁₁, R₁₂ and R₁₃, which may be identical or different, are chosen from aliphatic radicals, alicyclic radicals, arylaliphatic radicals having 1 to 20 carbon atoms, and lower hydroxyalkyl aliphatic radicals; or R₁₀, R₁₁, R₁₂ and R₁₃, which may be identical or different, form, together with the nitrogen atoms to which they are attached, heterocyclic rings optionally comprising a second heteroatom other than nitrogen; or R₁₀, R₁₁, R₁₂ and R₁₃, which may be identical or different, are chosen from linear and branched C₁-C₆ alkyl radicals substituted by at least one of nitriles, esters, acyls, amides, —CO—O—R₁₄-D groups, and —CO—NH—R₁₄-D groups, where R₁₄ is an alkylene and D is a quaternary ammonium group;

A₁ and B₁ are polymethylene groups having 2 to 20 carbon atoms chosen from linear and branched, saturated and unsaturated and which optionally comprise, bonded to or inserted into the main chain, at least one of aromatic rings oxygen atoms, sulphur atoms, sulphoxides, sulphones, disulphides, aminos, alkylaminos, hydroxyls, quaternary ammoniums, ureidos, amides, and ester groups; and

X⁻ is an anion derived from inorganic acids or organic acids; or

A₁, R₁₀ and R₁₂, together with the two nitrogen atoms to which they are attached, may form a piperazine ring. Additionally, if A₁ is a saturated or unsaturated, linear or branched alkylene or hydroxyalkylene radical, B₁ may also be a group —(CH₂)ₙ—CO—D-OC—(CH₂)ₙ— in which n ranges from 1 to 100, such as from 1 to 50, and D is:

a) a glycol residue of formula: —O-Z-O—, where Z is chosen from linear hydrocarbon radicals, branched hydrocarbon radicals, and groups chosen from one of the following formulae:

—(CH₂—CH₂—O)ₓ—CH₂—CH₂—

—[CH₂—CH(CH₃)—O]_{y}—CH₂—CH(CH₃)—

where x and y are integers chosen from 1 to 4, representinga defined and unique degree of polymerization or x and y can be any number chosen from 1 to 4, representing a mean degree of polymerization;

b) a disecondary diamine residue such as a piperazine derivative;

c) a diprimary diamine residue of formula: —NH—Y—NH—, where Y is chosen from linear hydrocarbon radicals, branched hydrocarbon radicals and divalent radicals

—CH₂—CH₂—S—S—CH₂—CH₂—;

d) at least one ureylene group of formula: —NH—CO—NH—;

X⁻ may be, for example, an anion such as chloride or bromide.

These polymers may have a number-average molecular mass generally ranging from 1000 to 100 000.

Polymers of this type are described for example in French Patents 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is possible in certain embodiments to use polymers which comprise repeating units of the following formula (XIV):

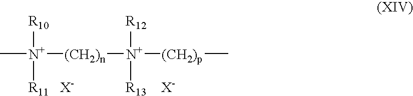

in which R₁₀, R₁₁, R₁₂ and R₁₃, which may be identical or different, are chosen from alkyl radicals having from 1 to 4 carbon atoms and hydroxyalkyl radicals having from 1 to 4 carbon atoms; n and p are integers ranging from 2 to 20; and X⁻ is an anion derived from an inorganic acids or organic acids.

(11) The quaternary polyammonium polymers comprising recurring units of formula (XV):

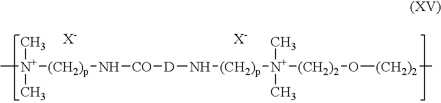

in which p is an integer ranging from 1 to 6 ; D may be zero or may be a group —(CH₂)ᵣ—CO— in which r is a number ranging from 4 to 7; and X⁻ is an anion.

Such polymers may be prepared, for example, according to the methods described in U.S. Pat. Nos. 4,157,388, 4,702,906, and 4,719,282. They are for example described in Patent Application EP-A-122 324.

Among these, mention may be made of the products sold under the names Mirapol® A 15, Mirapol® AD1, Mirapol® AZ1 and Mirapol® 175 by the company Miranol.

(12) Quaternary vinylpyrrolidone and vinylimidazole polymers such as, for example, the products sold under the names Luviquat® FC 905, FC 550, and FC 370 by the company B.A.S.F.

(13) Polyamines such as Polyquart H sold by Henkel, referenced under the name Polyethylene Glycol (15) Tallow Polyamine in the CTFA dictionary.

(14) The crosslinked polymers of methacryloyloxy(C₁-C₄ alkyl)tri(C₁-C₄ alkyl)ammonium salts such as the polymers obtained by homopolymerization of at least one dimethylaminoethyl methacrylate quaternized with at least one methyl chloride, or by copolymerization of at least one acrylamide with at least one dimethylaminoethyl methacrylate quaternized with at least one methyl chloride, the homo- or copolymerization being followed by crosslinking with at least one compound comprising olefinic unsaturation, for example methylenebisacrylamide. In certain embodiments, it is possible to employ a crosslinked acrylamide/methacryloyloxyethyltrimethylammonium chloride copolymer (20/80 by weight) in the form of a dispersion comprising 50% by weight of said copolymer in mineral oil. This dispersion is sold under the name of SALCARE® SC 92 by the company ALLIED COLLOIDS. It is also possible to employ a crosslinked methacryloyloxyethyltrimethylammonium chloride homopolymer comprising approximately 50% by weight of the homopolymer in mineral oil or in a liquid ester. These dispersions are sold under the names of SALCARE® SC 95 and SALCARE® SC 96 by the company ALLIED COLLOIDS.

Other cationic polymers that may be employed within the scope of the invention are polyalkyleneimines, in particular polyethyleneimines, polymers comprising vinylpyridine or vinylpyridinium units, condensates of polyamines and of epichlorohydrin, quaternary polyureylenes and chitin derivatives.

Among all the cationic polymers which may be used herein, mention may be made of polymers of the groups (1), (9), (10), (1 1) and (14) or, for example, polymers comprising recurring units of the following formulae (W) and (U):

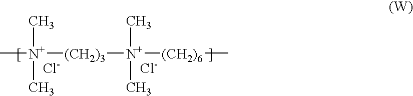

for example, those whose molecular weight, determined by gel permeation chromatography, ranges from 9500 to 9900; and

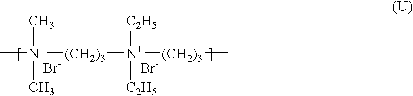

for example, those whose molecular weight, determined by gel permeation chromatography, is about 1200.

The concentration of additional cationic polymer(s) in the compositions disclosed herein may range from 0.01% to 10% by weight relative to the total weight of the composition, for example, from 0.05% to 5%, or for example from 0.1% to 3%.

Amphoteric Polymers

The amphoteric polymers which can be used in accordance with the present invention may be chosen from polymers comprising K and M units distributed statistically in the polymer chain wherein K is at least one unit which is derived from a monomer comprising at least one basic nitrogen atom and M is at least one unit which is derived from an acidic monomer comprising at least one group chosen from carboxylic groups and sulphonic groups; or alternatively K and M may be groups which are derived from zwitterionic monomers chosen from at least one of carboxybetaines and sulphobetaines;

K and M may also denote at least one cationic polymer chain comprising amine groups chosen from primary, secondary, tertiary and quaternary amine groups, in which at least one of the amine groups carries at least one group chosen from carboxylic groups and sulphonic groups linked via a hydrocarbon radical; or alternatively K and M may form part of a chain of a polymer with at least one α,β-dicarboxylic ethylene unit in which at least one of the carboxylic groups has been caused to react with at least one polyamine comprising at least one amine group chosen from primary and secondary amine groups.

For example, mention may be made of the amphoteric polymers corresponding to the definition given above and chosen from the following polymers:

1) The polymers resulting from the copolymerization of at least one monomer derived from at least one vinyl compound carrying at least one carboxylic group such as for example acrylic acid, methacrylic acid, maleic acid, and alpha-chloroacrylic acid, and of at least one basic monomer derived from at least one substituted vinyl compound comprising at least one basic atom, such as for example dialkylaminoalkyl methacrylate and acrylate, dialkylaminoalkylmethacrylamide and acrylamide. Such compounds are described, for example, in U.S. Pat. No. 3,836,537. Mention may also be made of the sodium acrylate/acryamidopropyltrimethylammonium chloride copolymer sold under the name POLYQUART KE® 3033 by the company HENKEL.

The vinyl compound may also be a dialkyldiallylammonium salt such as dimethyidiallylammonium chloride. The copolymers of acrylic and of the latter monomer are sold under the names MERQUAT® 280, MERQUAT® 295 and MERQUAT® PLUS 3330 by the company CALGON.

(2) The polymers comprising units which are derived from:

a) at least one monomer chosen from acrylamides and methacrylamides substituted on the nitrogen by an alkyl radical,

b) at least one acidic comonomer comprising at least one reactive carboxylic group, and

c) at least one basic comonomer such as esters with primary, secondary, tertiary and quaternary amine substituents of acrylic and methacrylic acids and the product of quaternization of dimethylaminoethyl methacrylate with at least one of dimethyl sulphate and diethyl sulphate.

The N-substituted acrylamides or methacrylamides may be, for example, groups whose alkyl radicals have 2 to 12 carbon atoms, for example N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers may be chosen, for example, from acrylic acids, methacrylic acids, crotonic acids, itaconic acids, maleic acids, fumaric acids, and alkyl monoesters having 1 to 4 carbon atoms of the group chosen from maleic anhydrides, fumaric anhydrides, maleic acids, and fumaricacids.

The basic comonomers may be, for example, methacrylates of aminoethyl, butylaminoethyl, N,N′-dimethylaminoethyl, and N-tert-butylaminoethyl.

Mention may be made of the copolymers whose CTFA name (4th ed. 1991) is Octylacrylamide/acrylates/butylaminoethylmethacrylate copolymer, such as the products sold under the names AMPHOMER® and LOVOCRYL® 47 by the company NATIONAL STARCH.

(3) The partially or completely alkylated and crosslinked polyaminoamides derived from polyaminoamides of general formula:

[CO—R₁₉—CO-Z] (XVI)

in which R₁₉ is a divalent radical derived from the group chosen from at least one of saturated dicarboxylic acids, mono- or dicarboxylic aliphatic acids with ethylenic double bonds, esters of a lower alkanol having 1 to 6 carbon atoms of these acids; or R₁₉ may be a radical which is derived from the addition of any one of said acids with an amine chosen from bis-primary amines and bis-secondary amines; and Z is a radical chosen from bis-primary, mono-secondary polyalkylene-polyamines, and bis-secondary polyalkylene-polyamines, and for example may represent:

a) in proportions ranging from 60 mol % to 100 mol %, the radical

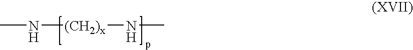

where x is 2 and p is chosen from 2 and 3, or alternatively x is 3 and p is 2. wherein this radical is derived from at least one of diethylenetriamine, triethylenetetraamine, and dipropylenetriamine;

b) in proportions ranging from 0 mol % to 40 mol %, the radical (XVII) above, in which x is 2 and p is 1 and which is derived from ethylenediamine, or the radical which is derived from piperazine:

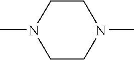

c) in proportions ranging from 0 mol % to 20 mol %, the radical —NH—(CH₂)₆—NH— which is derived from hexamethylenediamine, these polyaminoamines being crosslinked by adding at least one bifunctional crosslinking agent chosen from epihalohydrins, diepoxides, dianhydrides, and bis-unsaturated derivatives, by means of 0.025 mol to 0.35 mol of crosslinking agent per amine group of the polyaminoamide and alkylated by the action of at least one of acrylic acids, chloroacetic acids, alkanesultones, and their salts.

The saturated carboxylic acids may be chosen from acids having 6 to 10 carbon atoms, such as adipic acids, 2,2,4-trimethyladipic acids, 2,4,4-trimethyladipic acids, terephthalic acids, acids with an ethylene double bond such as, for example, acrylic acids, methacrylic acids, and itaconic acids.

The alkanesultones used in the alkylation may be, for example, propane or butanesultone, and the salts of the alkylating agents may be, for eample, sodium or potassium salts.

4) The polymers comprising zwitterionic units of formula:

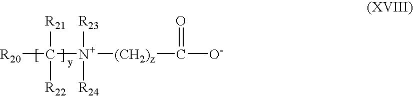

in which R₂₀ is chosen from polymerizable unsaturated groups such as acrylates, methacrylates, acrylamides and methacrylamides; y and z are integers chosen from 1 to 3; R₂₁ and R₂₂ are chosen from hydrogen atoms, methyls, ethyls, and propyls; R₂₃ and R₂₄ are chosen from hydrogen atoms and alkyl radicals such that the sum of the carbon atoms in R₂₃ and R₂₄ does not exceed 10.

The polymers comprising such units may also comprise units derived from nonzwitterionic monomers such as dimethyl acrylates, diethylaminoethyl acrylates, methacrylates, alkyl acrylates, alkyl methacrylates, acrylamides, methacrylamides, and vinyl acetates.

By way of example, mention may be made of copolymers of butyl methacrylate/dimethylcarboxymethylammonioethyl methacrylate, such as the product sold under the name DIAFORMER Z301® by the company SANDOZ.

(5) The polymers derived from chitosan comprising monomeric units of the following formulae (XIX), (XX), (XXI):

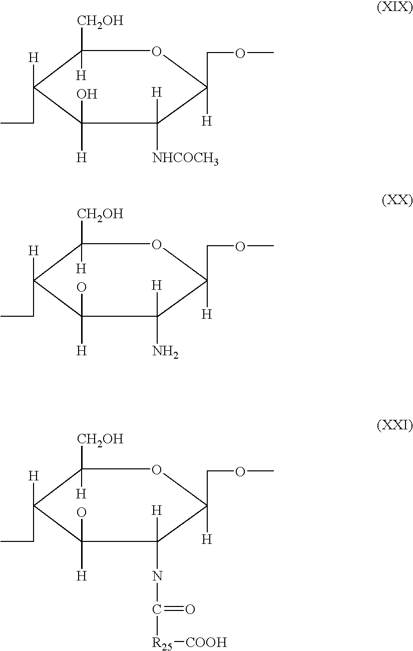

the (XIX) unit being present in proportions ranging from 0% to 30%, the (XX) unit being present in proportions ranging from 5% to 50%, and the (XXI) unit being present in proportions ranging from 30% to 90%, it being understood that in the (XXI) unit, R₂₅ is a radical of formula:

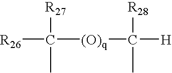

in which q is chosen from 0 and 1; and

if q is 0, R₂₆, R₂₇ and R₂₈, which may be identical or different, are chosen from hydrogen atoms, methyls, hydroxyls, acetoxy residues, amino residues, monoalkylamine residues, and dialkylamine residues, optionally interrupted by at least one nitrogen atom and optionally substituted with at least one group chosen from amine, hydroxyl, carboxyl, alkylthio, and sulphonic groups, and alkylthio residues whose alkyl group carries an amino residue, wherein at least one of R₂₆, R₂₇ and R₂₈ is a hydrogen atom;

or if q is 1, R₂₆, R₂₇ and R₂₈ are chosen from hydrogen atoms and the acid or base addition salts thereof.

(6) The polymers derived from the N-carboxyalkylation of chitosan such as N-carboxymethyl chitosan or N-carboxybutyl chitosan sold under the name EVALSAN® by the company JAN DEKKER.

(7) The polymers of the general formula (XXII) as described for example in French Patent 1,400,366:

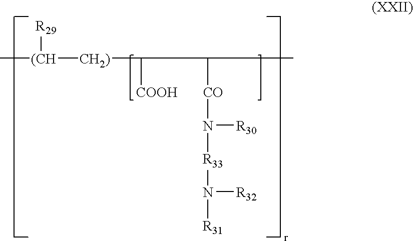

in which R₂₉ is chosen from hydrogen atoms, CH₃O radicals, CH₃CH₂O radicals, and phenyl radicals; R₃₀ is chosen from hydrogen atoms and lower alkyl radicals such as methyls and ethyls; R₃₁ is chosen from hydrogen atoms and lower alkyl radicals such as methyls and ethyls; R₃₂ is chosen from lower alkyl radicals such as methyls and ethyls and radicals of the formula: —R₃₃—N(R₃₁)₂; wherein R₃₃ is chosen from —CH₂—CH₂— groups, —CH₂—CH₂—CH₂— groups, and —CH₂—CH(CH₃)— groups, and wherein R₃₁ has the meanings mentioned above,

as well as the higher homologues of these radicals, having 6 or less carbon atoms;

r is such that the molecular weight ranges from 500 to 6 000 000 and, for example, ranges from 1000 to 1 000 000.

(8) Amphoteric polymers of the type -D-X-D-X— chosen from:

a) the polymers obtained by the action of chloroacetic acid or sodium chloroacetate on the compounds comprising at least one unit of formula:

-D-X-D-X-D- (XXIII)

where D is a radical

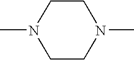

and X is chosen from the symbols E and E′; wherein E and E′, which may be identical or different, are at least one bivalent radical which is an alkylene radical with a linear or branched chain having 7 or less carbon atoms in the principal chain which is optionally substituted with hydroxyl groups and which may contain, in addition, at least one group chosen from oxygen atoms, nitrogen atoms, sulphur atoms, 1 to 3 aromatic rings, and 1-3 heterocyclic rings; wherein the oxygen, nitrogen and sulphur atoms may be present in the form of ether, thioether, sulphoxide, sulphone, sulphonium, alkylamine groups, alkenylamine groups, hydroxyl, benzylamine, amine oxide, quaternary ammonium, amide, imide, alcohol, ester and urethane groups;

b) The polymers of formula:

-D-X-D-X— (XXIV)

where D is a radical

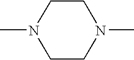

and X is chosen from the symbols E and E′ and, at least once, E′; wherein E has the meaning indicated above and E′ is a bivalent radical which is an alkylene radical with a linear or branched chain having 7 or less carbon atoms in the principal chain, which is optionally substituted with at least one hydroxyl radical and comprises at least one nitrogen atom, the nitrogen atom being substituted with an alkyl chain optionally interrupted by an oxygen atom and necessarily comprising at least one of the groups chosen from carboxyl functional groups and hydroxyl functional groups, and betainized by reaction with chloroacetic acid or sodium chloroacetate.

(9) The copolymers (C₁-C₅)alkyl vinyl ether/maleic anhydride partially modified by semiamidation with an N,N-dialkylaminoalkylamine such as N,N-dimethylaminopropylamine or by semiesterification with an N,N-dialkanolamine. These copolymers may also comprise other vinyl comonomers such as vinylcaprolactam.

In one embodiment, the amphoteric polymers used herein are those of the group (1).

According to certain embodiments of the invention, the additional amphoteric polymer(s)s may be present in an amount ranging from 0.01% to 10% by weight, for example, from 0.05% to 5% by weight, or for example, from 0.1% to 3% by weight, relative to the total weight of the composition.

The compositions of the invention may in certain embodiments comprise at least one surfactant.

The at least one surfactant may be chosen, alone or in the form of mixtures, from anionic, amphoteric, nonionic, zwitterionic and cationic surfactants.

The surfactants which may be suitable for carrying out the present invention are, for example, the following:

(i) Anionic Surfactants:

By way of example of anionic surfactants which can be used, alone or as mixtures, in the context of the present invention mention may be made of, as non-limiting examples, the salts, such as alkali metal, for example sodium, salts, ammonium salts, amine salts, amino alcohol salts, and magnesium salts, of the following compounds: alkyl sulphates, alkyl ether sulphates, alkylamido ether sulphates, alkylarylpolyether sulphates, monoglyceride sulphates, alkyl sulphonates, alkyl phosphates, alkylamidesulphonates, alkyl aryl sulphonates, α-olefinsulphonates, paraffinsulphonates, (C₆-C₂₄)alkyl sulphosuccinates, (C₆-C₂₄)alkyl ether sulphosuccinates, (C₆-C₂₄)alkylamide sulpho-succinates, (C₆-C₂₄)alkyl sulphoacetates, (C₆-C₂₄)acyl sarcosinates, and (C₆-C₂₄)acyl glutamates. It is also possible to use (C₆-C₂₄)alkyl polyglycoside carboxylic esters, such as alkyl glucoside citrates, alkyl polyglycoside tartrates, and alkyl polyglycoside sulpho-succinates, alkyl sulphosuccinamates, acyl isethionates and N-acyltaurates, the alkyl or acyl radicals of all these various compounds, for example those comprising 12 to 20 carbon atoms, and the aryl radicals, for example, may be phenyl groups and benzyl groups. Among the anionic surfactants which can be used, mention may also be made of the salts of fatty acids such as the salts of oleic, ricinoleic, palmitic and stearic acids, the acids of copra oil and of hydrogenated copra oil; the acyllactylates whose acyl radical comprises 8 to 20 carbon atoms. It is also possible to use the alkyl D-galactoside uronic acids and their salts, the polyoxyalkylenated (C₆-C₂₄)alkyl ether carboxylic acids, the polyoxyalkylenated (C₆-C₂₄)alkylaryl ether carboxylic acids, the polyoxyalkylenated (C₆-C₂₄)alkyl amido ether carboxylic acids and salts thereof, for example, those comprising 2 to 50 carbon alkylenes, such as ethylene, oxide groups, and mixtures thereof.

(ii) Nonionic Surfactant(s):

The nonionic surfactants themselves are also compounds which may be well known per se (in this respect see especially the “Handbook of Surfactants” by M. R. Porter, published by Blackie & Son (Glasgow and London), 1991, pp. 116-178) and, in the context of the present invention, their nature does not necessarily assume any particular character. They may thus be chosen from, as non-limiting examples, alpha-diols, polyethoxylated alkylphenols, and polypropoxylated alkylphenols, which have at least one fatty chain comprising, for example, 8 to 18 carbon atoms, and it being possible for the number of ethylene oxide or propylene oxide groups to range, for example, from 2 to 50. Mention may also be made of the copolymers of ethylene oxide and propylene oxide and the condensates of ethylene oxide and propylene oxide with fatty alcohols; the polyethoxylated fatty amides comprising, for example, 2 to 30 mol of ethylene oxide, the polyglycerolated fatty amides comprising on average 1 to 5 glycerol groups, for example 1.5 to 4 glycerol groups; the oxyethylenated fatty acid esters of sorbitan comprising 2 to 30 mol of ethylene oxide; the fatty acid esters of sucrose, the fatty acid esters of polyethylene glycol, alkylpoly-glycosides, N-alkylglucamine derivatives, amine oxides such as oxides of (C₁₀-C₁₄)-alkylamines and N-acylaminopropylmorpholine oxides.

(iii) Amphoteric or Zwitterionic Surfactants:

The amphoteric or zwitterionic surfactants, the nature of which is not necessarily of particular importance in the context of the present invention, may be, as non-limiting examples, derivatives of aliphatic secondary or tertiary amines in which the aliphatic radical is a linear or branched chain comprising 8 to 18 carbon atoms and comprising at least one water-solubilizing anionic group, such as, for example, carboxylates, sulphonates, sulphates, phosphates, and phosphonate. (C₈-C₂₀)alkylbetaines, sulphobetaines, (C₈-C₂₀)alkylamido(C₁-C₆)alkylbetaines, and (C₈-C₂₀)alkylamido(C₁-C₆)alkylsulphobetaines may also be mentioned.

Among the amine derivatives, mention may be made of the products sold under the name MIRANOL, as described in U.S. Pat. Nos. 2,528,378 and 2,781,354 and classified in the CTFA dictionary, 3rd edition, 1982, under the names Amphocarboxyglycinates and Amphocarboxypropionates having the respective structures:

R₂—CONHCH₂CH₂—N(R₃)(R₄)(CH₂COO⁻)

in which: R₂ is chosen from alkyl radicals of an acid R₂—COOH present in hydrolysed copra oil, heptyl radicals, nonyl radicals, and undecyl radicals; R₃ is a beta-hydroxyethyl group; and R₄ is a carboxymethyl group;

and

R₂′—CONHCH₂CH₂—N(B)(C)

in which:

B is —CH₂CH₂OX′; C is —(CH₂)_{z}—Y′, wherein z is chosen from 1 and 2;

X′ is chosen from —CH₂CH₂—COOH groups and hydrogen atoms;

Y′ is chosen from —COOH radicals and —CH₂—CHOH—SO₃H radicals;

R₂′ is chosen from alkyl radicals of an acid R₉—COOH present in copra oil or hydrolysed linseed oil, alkyl radicals, for example C₇, C₉, C₁₁ and C₁₃ alkyl radicals C₁₇ alkyl radicals, their iso forms, and unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylampho-dipropionate, Disodium Capryloamphodipropionate, Lauro-amphodipropionic acid, and Cocoamphodipropionic acid.

By way of example, mention may be made of the cocoamphodiacetate sold under the trade name MIRANOL® C2M concentrated by the company RHODIA CHIMIE.

(iv) Cationic Surfactants:

Among the cationic surfactants, mention may be made of, as non-limiting examples: the salts of optionally polyoxyalkylenated amines chosen from primary, secondary, and tertiary amines; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives and amine oxides of a cationic nature.

The quantities of surfactants present in the composition according to certain embodiments may range from 0.01% to 40%, for example from 0.5% to 30%, by weight relative to the total weight of the composition.

Additional Thickening Agents

The compositions disclosed herein may also comprise other rheology adjusting agents, such as cellulosic thickeners (such as hydroxyethylcellulose, hydroxy-propylcellulose, carboxymethylcellulose, and the like), guar gum and its derivatives (such as hydroxypropylguar and the like), gums of microbial origin (such as xanthan gum, scleroglucan gum, and the like), synthetic thickeners such as crosslinked homopolymers of acrylic acid and acrylamidopropanesulphonic acid and ionic or nonionic associative polymers such as the polymers sold under the names PEMULEN® TR1 and TR2 by the company GOODRICH, the polymers sold under the name SALCARE® SC90 by the company ALLIED COLLOIDS, the polymers sold under the names ACULYN® 22, 28, 33, 44 and 46 by the company ROHM & HAAS, and the polymers sold under the names ELFACOS® T210 and T212 by the company AKZO.

These supplementary thickeners may be present in an amount ranging from 0.01% to 10% by weight relative to the total weight of the composition.

The medium suitable for dyeing for the composition is, for example, an aqueous medium comprising water and may, for example, comprise, cosmetically acceptable organic solvents including for example alcohols such as ethyl alcohol, isopropyl alcohol, benzyl alcohol and phenylethyl alcohol, glycols and glycol ethers such as, for example, monomethyl, monoethyl and monobutyl ethers of ethylene glycol, propylene glycol or its ethers, such as, for example, monomethyl ether of propylene glycol, butylene glycol, and dipropylene glycol, as well as the alkyl ethers of diethylene glycol such as for example monoethyl ether and monobutyl ether of diethylene glycol. The solvents may then be present in concentrations ranging from 0.5% to 20%, for example from 2% to 10%, by weight relative to the total weight of the composition.

The composition A may also comprise an effective quantity of other agents, which may be previously known in oxidation dyeing, such as various customary adjuvants such as sequestrants such as EDTA and etidronic acid, UV-screening agents, waxes, volatile and nonvolatile silicones which may be cyclic, linear, or branched, organomodified, for example with amine groups, or otherwise, preservatives, ceramides, pseudoceramides, vegetable, mineral and synthetic oils, vitamins and provitamins such as panthenol, opacifiers, associative polymers other than those of the present invention, and for example nonionic associative polyether-polyurethanes.

The composition may also comprise reducing agents and antioxidants. These may be, for example, chosen from sodium sulphites, thioglycolic acids, thiolactic acids, sodium bisulphites, dehydroascorbic acids, hydroquinones, 2-methylhydroquinones, tert-butylhydroquinones, and homogentisic acids, and they may generally be present in quantities ranging from 0.05% to 3% by weight relative to the total weight of the composition.

Of course persons skilled in the art will be careful to choose the possible additional compound or compounds mentioned above so that the advantageous properties intrinsically attached to the dyeing composition according to the invention are not, or substantially not, impaired by the envisaged addition or additions.

In the ready-to-use composition or in the composition B, the at least one oxidizing agent is preferably chosen from urea peroxide, alkali metal bromates and ferri-cyanides, persalts such as perborates and persulphates. Mention may be made, for example, of the use of hydrogen peroxide. This oxidizing agent may comprise a solution of hydrogen peroxide whose titre may range, for example, from 1 to 40 volumes, or, for example, from 5 to 40 volumes.

It is also possible to use as oxidizing agent one or more oxidation-reduction enzymes such as laccases, peroxidases and oxidoreductases containing 2 electrons, such as uricase, where appropriate in the presence of their respective donor or cofactor.

The pH of the ready-to-use compositions applied to the keratin fibers [such as the composition resulting from the mixture of the dyeing composition A and of the oxidizing composition B and optionally of the composition C], generally ranges from 4 to 11. It may range, for example, from 6 to 10, and may be adjusted to the desired value by means of acidifying or alkalinizing agents well known in the state of the art for dyeing keratin fibers.

Among the alkalinizing agents, mention may be made by way of example of aqueous ammonias, alkali metal carbonates, alkanolamines such as mono-, di- and triethanolamines as well their derivatives, oxyethylenated ethylenediamines, oxypropylenated ethylenediamines, oxyethylenated hydroxyalkylamines, oxypropylenated hydroxyalkylamines, sodium hydroxides, potassium hydroxides, and compounds having the following formula (XXV):

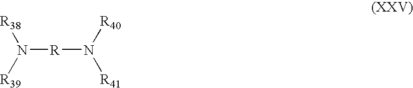

in which R is chosen from propylene residues, optionally substituted with hydroxyl groups, and C₁-C₄ alkyl radicals; R₃₈, R₃₉, R₄₀ and R₄₁, which may be identical or different, are chosen from hydrogen atoms, C₁-C₄ alkyl radicals, and C₁-C₄ hydroxyalkyl radicals.

The acidifying agents may be conventionally, by way of example, inorganic acids and organic acids, such as hydrochloric acid, orthophosphoric acid, carboxylic acids such as tartaric acid, citric acid, lactic acid and sulphonic acids.

The dyeing method according to the invention may, for example, comprise applying the ready-to-use composition, prepared at the time of use from the compositions A and B and optionally C described above, to the dry or wet keratin fibers, and in allowing it to act for an exposure time ranging, for example, from 1 to 60 minutes, or for example from 10 to 45 minutes, in rinsing the fibers, and optionally in washing them with shampoo, and then in rinsing them again, and in drying them.

According to said methods, the compositions A and/or B may additionally comprise at least one additional cationic or amphoteric polymer and at least one surfactant.

A concrete example illustrating the invention is indicated below, without however exhibiting a limiting character.

### EXAMPLE

The following compositions were prepared:

(quantities expressed in grams)

Oxidizing Composition (in Grams):

| | |
|---|---|
| Cetylstearyl alcohol (80%)/cetylstearyl alcohol containing | 2.35 |
| 30 EO (20%) mixture (SINNOWAX AO from COGNIS) | |
| Oleic acid diethanolamine | 0.95 |
| Glycerin | 0.5 |
| Hydrogen peroxide as a 50% solution in water | 12 |
| Sequestering agent | 0.15 |
| Stabilizing agents | 0.125 |
| Perfume | qs |
| Acidifying agents | qs pH 2.8 |
| Demineralized water | qs 100 |

Dyeing Composition (in Grams):

| | |
|---|---|
| Natural lauric acid | 2.5 |
| Oxyethylenated lauryl alcohol (12 EO) | 7.5 |
| Cetylstearyl alcohol (C16/C18 50/50) | 10 |
| Glycol monostearate | 2 |
| Oxyethylenated oleocetyl alcohol (30 EO) | 3 |
| Oxyethylenated decyl alcohol (3 EO) | 10 |
| Pyrogenic silica with a hydrophobic character | 1 |
| Pure monoethanolamine | 1.2 |
| Dimethyl diallyl ammmonium chloride homopolymer as a 40% | 7 |
| aqueous solution | |
| Propylene glycol | 10 |
| Terpolymer of vinylpyrrolidone, dimethyl- | 4 |
| aminopropylmethacrylamide and lauryldimethyl- | |
| propylmethacrylamidoammonium chloride (74/15/11) | |
| Crosslinked polyacrylic acid | 0.4 |
| Diethylenetriaminepentaacetic acid, pentasodium salt as a | 2 |
| 40% aqueous solution | |
| Ammonium thiolactate as a 58% aqueous solution (50% as | 0.8 |
| thiolactic acid) | |
| Mono-tert-butylhydroquinone | 0.3 |
| Para-toluenediamine sulphate | 5 |
| 1,3-Dihydroxybenzene (resorcinol) | 0.6 |
| 1-hydroxy-3-aminobenzene | 0.4 |
| 1-(β-hydroxyethyloxy)-2,4-diaminobenzene dihydrochloride | 0.8 |
| Aqueous ammonia (at 20.5% of ammonia) | 10 |

What is claimed is:

1. A composition for the oxidation dyeing of keratin fibers comprising, in a medium suitable for dyeing, at least one oxidation dye in the form of a sulphate salt, and at least one cationic poly(vinyllactam) polymer comprising:
a) at least one monomer chosen from vinyllactams and alkylvinyllactams; and
b) at least one monomer chosen from the following formulas (Ia) and (Ib): 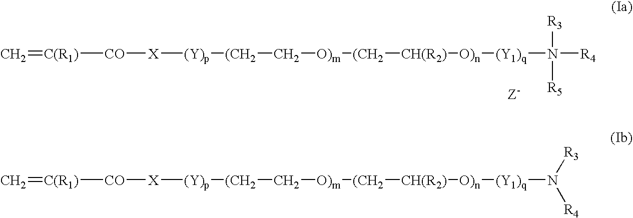
in which:
X is chosen from oxygen atoms and NR₆ radicals;
R₁ and R₆, which may be identical or different, are chosen from hydrogen atoms, linear C₁-C₅ alkyl radicals, and branched C₁-C₅ alkyl radicals;
R₂ is chosen from linear C₁-C₄ alkyl radicals and branched C₁-C₄ alkyl radicals;
R₃, R₄ and R₅, which may be identical or different, are chosen from hydrogen atoms, linear C₁-C₃₀ alkyl radicals, branched C₁-C₃₀ alkyl radicals, and radicals of formula (II):
—(Y₂)ᵣ—(CH₂—CH(R₇)—O)ₓ—R₈ (II)
in which:
Y, Y₁ and Y₂ , which may be identical or different, are chosen from linear C₂-C₁₆ alkylene radicals and branched C₂-C₁₆ alkylene radicals;
R₇ is chosen from hydrogen atoms, linear C₁-C₄ alkyl radicals, branched C₁-C₄ alkyl radicals, linear C₁-C₄ hydroxyalkyl radical, and branched C₁-C₄ hydroxyalkyl radicals;
R₈ is chosen from hydrogen atoms, linear C₁-C₃₀ alkyl radicals, and branched C₁-C₃₀ alkyl radicals;
p, q and r which may be identical or different, are chosen from 0 and 1;
m and n, which may be identical or different, are chosen from integers ranging from 0 to 100;
x is an integer ranging from 1 to 100,
Z⁻ is chosen from organic acid anions and inorganic acid anions, with the provisos that:
at least one of R₃, R₄, R₅ and R₈ is chosen from linear C₉-C₃₀ alkyl radicals and branched C₉-C₃₀ alkyl radicals,
if either m or n is different from 0, then q is 1,
if either m or n is 0, then either p or q is 0.

2. The composition according to claim 1, wherein the keratin fibers are hair.

3. The composition according to claim 1, wherein the oxidation dye in the form of a sulphate salt is present in a concentration of at least 2% by weight relative to the total weight of the composition.

4. The composition according to claim 1, wherein the at least one monomer chosen from vinyllactams and alkylvinyllactams is a monomer of formula (III): 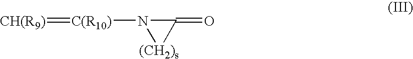
in which:
s is an integer ranging from 3 to 6;
R₉ is chosen from hydrogen atoms and C₁-C₅ alkyl radicals;
R₁₀ is chosen from hydrogen atoms and C₁-C₅ alkyl radicals;
with the proviso that at least one of R₉ and R₁₀ is a hydrogen atom.

5. The composition according to claim 4, wherein the monomer of formula (III) is vinylpyrrolidone.

6. The composition according to claim 1, wherein R₃, R₄ and R₅, which may be identical or different, are chosen from hydrogen atoms, linear C₁-C₃₀ alkyl radicals, and branched C₁-C₃₀ alkyl radicals.

7. The composition according to claim 1, wherein the at least one monomer chosen from the formulas (Ia) and (Ib) is a monomer of formula (Ia).

8. The composition according to claim 7, wherein in the formula (Ia), m and n are 0.

9. The composition according to claim 1, wherein, in the monomer of formula (Ia), Z⁻ is chosen from halide ions, phosphate ions, methosulphate ions, and tosylate ions.

10. The composition according to claim 1, wherein the at least one cationic poly(vinyllactam) polymer further comprises at least one additional monomer chosen from cationic monomers and nonionic monomers.

11. The composition according to claim 10, wherein the at least one cationic poly(vinyllactam) polymer is a terpolymer comprising:
a) at least one monomer (a) of formula (III);
b) at least one monomer (b) of formula (Ia) in which p is 1, q is 0, R₃ and R₄, which may be identical or different, are chosen from hydrogen atoms and C₁-C₅ alkyl radicals, and R₅ is a C₉-C₂₄ alkyl radical; and
c) at least one monomer (c) of formula (Ib) in which R₃ and R₄, which may be identical or different, are chosen from hydrogen atoms and C₁-C₅ alkyl radicals.

12. The composition according to claim 11, wherein the terpolymer comprises 40% to 95% of the at least one monomer (a), 0.25% to 50% of the at least one monomer (b), and 0.1% to 55% of the at least one monomer (c), by weight relative to the total weight of the terpolymer.

13. The composition according to claim 1, wherein the at least one cationic poly(vinyllactam) polymer is chosen from the following terpolymers:
vinylpyrrolidone/dimethylaminopropylmethacrylamide/dodecyldimethylmethacryl-amidopropylammonium tosylate,
vinylpyrrolidone/dimethylaminopropylmethacrylamide/cocoyldimethyl-methacrylamidopropylammonium tosylate,
vinylpyrrolidone/dimethylaminopropylmethacrylamide/lauryldimethylmethacrylamido-propylammonium tosylate, and
vinylpyrrolidone/dimethylaminopropylmethacrylamide/lauryldimethylmethacrylamido-propylammonium chloride.

14. The composition according to claim 1, wherein the weight-average molecular mass of the at least one cationic poly(vinyllactam) ranges from 500 to 20,000,000.

15. The composition according to claim 14, wherein the weight-average molecular mass of the at least one cationic poly(vinyllactam) ranges from 200,000 to 2,000,000.

16. The composition according to claim 15, wherein the weight-average molecular mass of the at least one cationic poly(vinyllactam) ranges from 400,000 to 8,000,000.

17. The composition according to claim 1, wherein the at least one cationic poly(vinyllactam) is present in an amount ranging from 0.01% to 10% by weight relative to the total weight of the composition.

18. The composition according to claim 17, wherein the at least one cationic poly(vinyllactam) is present in an amount ranging from 0.1% to 5% by weight relative to the total weight of the composition.

19. The composition according to claim 1, wherein the at least one oxidation dye, in the form of a sulphate salt is chosen from
sulphate salts of oxidation bases chosen from para-phenylenediamines, double bases, ortho-aminophenols, para-aminophenols, and heterocyclic bases, and
sulphate salts of couplers chosen from meta-aminophenols, meta-phenylenediamines, meta-diphenols, naphthols, and heterocylic couplers.

20. The composition according to claim 19, wherein the at least one oxidation dye in the form of a sulphate salt is chosen from para-toluenediamine sulphates, N,N-bis(β-hydroxyethyl)-para-phenylenediamine sulphates, 2-(β-hydroxyethyl)-para-phenylenediamine sulphates, N-methyl-para-aminophenol hemisulphates, 1-(β-hydroxy-ethyl)-4,5-diaminopyrazole sulphates, and 4-(β-hydroxyethyl)amino-2-aminoanisole sulphates.

21. The composition according to claim 1, wherein the at least one oxidation dye in the form of a sulphate salt is present in an amount ranging from 2% to 25% by weight relative to the total weight of the composition.

22. The composition according to claim 21, wherein the at least one oxidation dye in the form of a sulphate salt is present in an amount ranging from 2.25% to 15% by weight relative to the total weight of the composition.

23. The composition according to claim 22, wherein the at least one oxidation dye in the form of a sulphate salt is present in an amount ranging from 2.5% to 10% by weight relative to the total weight of the composition.

24. The composition according to claim 1, further comprising at least one additional oxidation dye, other than the at least one oxidation dye in the form of a sulphate salt, chosen from at least one oxidation base and at least one coupler.

25. The composition according to claim 24, wherein the at least one additional oxidation dye is at least one oxidation base.

26. The composition according to claim 24, wherein the at least one oxidation base is chosen from para-phenylenediamines, double bases, ortho-aminophenols, para-aminophenols, heterocyclic bases, and their acid addition salts other than sulphates.

27. The composition according to claim 24, wherein the at least one additional oxidation base is present in an amount ranging from 0.0005% to 20% by weight relative to the total weight of the composition.

28. The composition according to claim 24, wherein the at least one coupler is chosen from meta-phenylenediamines, meta-aminophenols, meta-diphenols, heterocyclic couplers, and their acid addition salts other than sulphates.

29. The composition according to claim 24, wherein the at least one coupler is present in an amount ranging from 0.0001% to 20% by weight relative to the total weight of the composition.

30. The composition according to claim 26, wherein the acid addition salts are chosen from hydrochlorides, hydrobromides, tartrates, lactates, and acetates.

31. The composition according to claim 28, wherein the acid addition salts are chosen from hydrochlorides, hydrobromides, tartrates, lactates, and acetates.

32. The composition according to claim 1, further comprising at least one direct dye.

33. The composition according to claim 1, further comprising at least one polymer chosen from amphoteric polymers and cationic polymers different from said at least one cationic poly(vinyllactam).

34. The composition according to claim 33, wherein the cationic polymer is a quaternary polyammonium comprising recurring units of the formula (W):

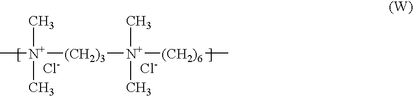

35. The composition according to claim 33, wherein the cationic polymer is a quaternary polyammonium comprising recurring units of the formula (U):

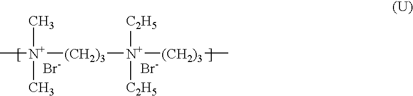

36. The composition according to claim 33, wherein the amphoteric polymer is a copolymer comprising monomers of at least one acrylic acid and at least one salt of dimethyldiallylammonium.

37. The composition according to claim 33, wherein the at least one polymer is present in an amount ranging from 0.01% to 10% by weight relative to the total weight of the composition.

38. The composition according to claim 37, wherein the at least one polymer is present in an amount ranging from 0.05% to 5%, by weight relative to the total weight of the composition.

39. The composition according to claim 38, wherein the wherein the at least one polymer is present in an amount ranging from 0.1% to 3% by weight relative to the total weight of the composition.

40. The composition according to claim 1, further comprising at least one surfactant chosen from anionic, cationic, nonionic, and amphoteric surfactants.

41. The composition according to claim 40, wherein the at least one surfactant is present in an amount ranging from 0.01% to 40% by weight relative to the total weight of the composition.

42. The composition according to claim 41, wherein the at least one surfactant is present in an amount ranging from 0.5% to 30% by weight relative to the total weight of the composition.

43. The composition according to claim 1, further comprising at least one thickening agent.

44. The composition according to claim 1, further comprising at least one reducing agent, in quantities ranging from 0.05% to 3% by weight relative to the total weight of the composition.

45. The composition according to claim 1, further comprising at least one oxidizing agent and wherein the composition is ready for use.

46. The composition according to claim 45, wherein the at least one oxidizing agent is chosen from hydrogen peroxide, urea peroxide, alkali metal bromates, alkali metal ferricyanides, persalts, and oxidation-reduction enzymes optionally with the respective donor or cofactor.

47. The composition according to claim 46, wherein the at least one oxidizing agent is hydrogen peroxide.

48. The composition according to claim 47, wherein the at least one oxidizing agent is a hydrogen peroxide solution whose titre ranges from 1 to 40 volumes.

49. The composition according to claim 45, wherein the pH ranges from 4 to 11.

50. A method for dyeing keratin fibers comprising
applying to the fibers at least one composition A comprising, in a medium suitable for dyeing, at least one oxidation dye in the form of a sulphate salt,
developing the color at a pH chosen from alkaline, neutral and acidic with the aid of a composition B comprising at least one oxidizing agent, wherein composition B is mixed with composition A at the time of use, or applied sequentially to the composition A without intermediate rinsing,
wherein at least one cationic poly(vinyllactam) is present in the composition A, in the composition B or in both compositions A and B, said at least one cationic poly(vinyllactam) comprising:
a) at least one monomer chosen from vinyllactams and alkylvinyllactams; and
b) at least one monomer chosen from the following formulas (Ia) and (Ib): 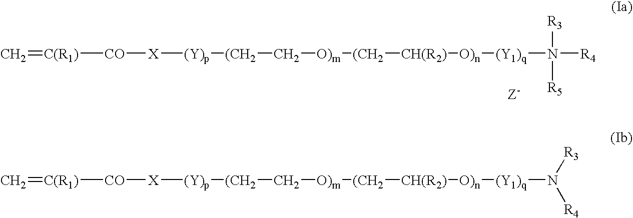
in which:
X is chosen from oxygen atoms and NR₆ radicals;
R₁ and R₆, which may be identical or different, are chosen from hydrogen atoms, linear C₁-C₅ alkyl radicals, and branched C₁-C₅ alkyl radicals;
R₂ is chosen from linear C₁-C₄ alkyl radicals and branched C₁-C₄ alkyl radicals;
R₃, R₄ and R₅, which may be identical or different, are chosen from hydrogen atoms, linear C₁-C₃₀ alkyl radicals, branched C₁-C₃₀ alkyl radicals, and radicals of formula (II):
—(Y₂)ᵣ—(CH₂—CH(R₇)—O)_{X}—R₈ (II)
in which:
Y, Y₁ and Y₂ , which may be identical or different, are chosen from linear C₂-C₁₆ alkylene radicals and branched C₂-C₁₆ alkylene radicals;
R₇ is chosen from hydrogen atoms, linear C₁-C₄ alkyl radicals, branched C₁-C₄ alkyl radicals, linear C₁-C₄ hydroxyalkyl radical, and branched C₁-C₄ hydroxyalkyl radicals;
R₈ is chosen from hydrogen atoms, linear C₁-C₃₀ alkyl radicals, and branched C₁-C₃₀ alkyl radicals;
p, q and r which may be identical or different, are chosen from 0 and 1;
m and n, which may be identical or different, are chosen from integers ranging from 0 to 100;
x is an integer ranging from 1 to 100,
Z₋ is chosen from organic acid anions and inorganic acid anions, with the provisos that:
at least one of R₃, R₄, R₅ and R₈ is chosen from linear C₉-C₃₀ alkyl radicals and branched C₉-C₃₀ alkyl radicals,
if either m or n is different from 0, then q is 1,
if either m or n is 0, then either p or q is 0.

51. The method according to claim 50, wherein the keratin fibers are hair.

52. The method according to claim 50, wherein the at least one oxidation dye in the form of a sulphate salt is present in a concentration of at least 2% by weight relative to the total weight of the composition.

53. The method according to claim 50, further comprising applying a ready to use composition, prepared at the time of use from the compositions A and B, to the keratin fibers, allowing it to act for an exposure time ranging from 1 to 60 minutes, rinsing the fibers, and then optionally washing the fibers with shampoo, rinsing the fibers again, and drying the fibers.

54. A two-compartment device for dyeing keratin fibers comprising
a first compartment comprises a composition A1 comprising, in a medium suitable for dyeing, at least one oxidation dye in the form of a sulphate salt, and
a second compartment comprises a composition B1 comprising, in a medium suitable for dyeing, at least one oxidizing agent, and
wherein at least one cationic poly(vinyllactam) is present in the composition A1, in the composition B1 or in both compositions A1 and B1, said at least one cationic poly(vinyllactam) comprising:
a) at least one monomer chosen from vinyllactams and alkylvinyllactams; and
b) at least one monomer chosen from the following formulas (Ia) and (Ib): 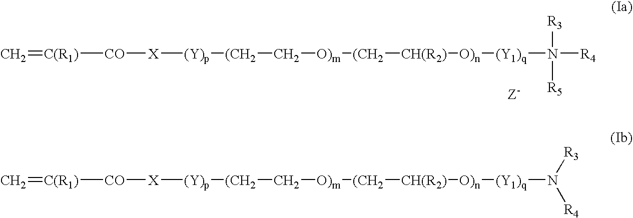
in which:
X is chosen from oxygen atoms and NR₆ radicals;
R₁ and R₆, which may be identical or different, are chosen from hydrogen atoms, linear C₁-C₅ alkyl radicals, and branched C₁-C₅ alkyl radicals;
R₂ is chosen from linear C₁-C₄ alkyl radicals and branched C₁-C₄ alkyl radicals;
R₃, R₄ and R₅, which may be identical or different, are chosen from hydrogen atoms, linear C₁-C₃₀ alkyl radicals, branched C₁-C₃₀ alkyl radicals, and radicals of formula (II):
—(Y₂)ᵣ—(CH₂—CH(R₇)—O)ₓ—R₈ (II)
in which:
Y, Y₁ and Y₂ , which may be identical or different, are chosen from linear C₂-C₁₆ alkylene radicals and branched C₂-C₁₆ alkylene radicals;
R₇ is chosen from hydrogen atoms, linear C₁-C₄ alkyl radicals, branched C₁-C₄ alkyl radicals, linear C₁-C₄ hydroxyalkyl radical, and branched C₁-C₄ hydroxyalkyl radicals;
R₈ is chosen from hydrogen atoms, linear C₁-C₃₀ alkyl radicals, and branched C₁-C₃₀ alkyl radicals;
p, q and r which may be identical or different, are chosen from 0 and 1;
m and n, which may be identical or different, are chosen from integers ranging from 0 to 100;
x is an integer ranging from 1 to 100,
Z⁻ is chosen from organic acid anions and inorganic acid anions, with the provisos that:
at least one of R₃, R₄, R₅ and R₈ is chosen from linear C₉-C₃₀ alkyl radicals and branched C₉-C₃₀ alkyl radicals,
if either m or n is different from 0, then q is 1,
if either m or n is 0, then either p or q is 0.

55. The two-compartment device according to claim 54, wherein the keratin fibers are hair.

56. The two-compartment device according to claim 54, wherein the at least one oxidation dye in the form of a sulphate salt is present in a concentration of at least 2% by weight relative to the total weight of the composition.

57. A three-compartment device for dyeing keratin fibers, comprising
a first compartment comprises a composition A2 comprising, in a medium suitable for dying, at least one oxidation dye in the form of a sulphate salt,
a second compartment comprising a composition B2 comprising, in a medium suitable for dyeing, at least one oxidizing agent, and
a third compartment comprising a composition C comprising, in a medium suitable for dyeing, at least one cationic poly(vinyllactam),
it being possible for the composition A2, the composition B2, or both compositions A2 and B2 to also comprise said at least one cationic poly(vinyllactam),
wherein said at least one cationic poly(vinyllactam) comprises:
a) at least one monomer chosen from vinyllactams and alkylvinyllactams; and
b) at least one monomer chosen from the following formulas (Ia) and (Ib): 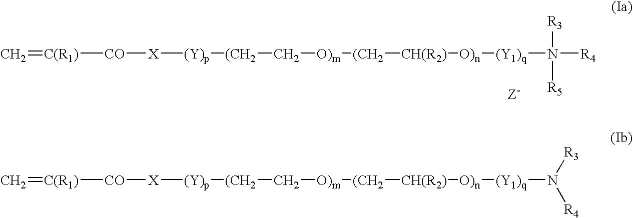
in which:
X is chosen from oxygen atoms and NR₆ radicals;
R₁ and R₆, which may be identical or different, are chosen from hydrogen atoms, linear C₁-C₅ alkyl radicals, and branched C₁-C₅ alkyl radicals;
R₂ is chosen from linear C₁-C₄ alkyl radicals and branched C₁-C₄ alkyl radicals;
R₃, R₄ and R₅, which may be identical or different, are chosen from hydrogen atoms, linear C₁-C₃₀ alkyl radicals, branched C₁-C₃₀ alkyl radicals, and radicals of formula (II):
—(Y₂)ᵣ—(CH₂—CH(R₇)—O)ₓ—R₈ (II)
in which:
Y, Y₁ and Y₂ , which may be identical or different, are chosen from linear C₂-C₁₆ alkylene radicals and branched C₂-C₁₆ alkylene radicals;
R₇ is chosen from hydrogen atoms, linear C₁-C₄ alkyl radicals, branched C₁-C₄ alkyl radicals, linear C₁-C₄ hydroxyalkyl radical, and branched C₁-C₄ hydroxyalkyl radicals;
R₈ is chosen from hydrogen atoms, linear C₁-C₃₀ alkyl radicals, and branched C₁-C₃₀ alkyl radicals;
p, q and r which may be identical or different, are chosen from 0 and 1;
m and n, which may be identical or different, are chosen from integers ranging from 0 to 100;
x is an integer ranging from 1 to 100,
Z⁻ is chosen from organic acid anions and inorganic acid anions,
with the provisos that:
at least one of R₃, R₄, R₅ and R₈ is chosen from linear C₉-C₃₀ alkyl radicals and branched C₉-C₃₀ alkyl radicals,
if either m or n is different from 0, then q is 1,
if either m or n is 0, then either p or q is 0.

58. The three-compartment device according to claim 57, wherein the keratin fibers are hair.

59. The three-compartment device according to claim 57, wherein the at least one oxidation dye in the form of a sulphate salt is present in a concentration of at least 2% by weight relative to the total weight of the composition.
